(19) United States
(12) Patent Application Publication
(10) Pub. No.: US 2016/0095555 A1
(43) Pub. Date: Apr. 7, 2016

(54) GRAPHICAL TECHNIQUE FOR DETECTING CONGESTIVE HEART FAILURE

(71) Applicant: TOSENSE, INC., La Jolla, CA (US)

(72) Inventors: Derek Gregory STAINER, San Diego, CA (US); Samuel Anthony WEISER, Los Alamitos, CA (US); Matthew BANET, San Diego, CA (US)

(21) Appl. No.: 14/877,694

(22) Filed: Oct. 7, 2015

Related U.S. Application Data
(60) Provisional application No. 62/060,926, filed on Oct. 7, 2014.

(51) Int. Cl. A61B 5/00; A61B 5/145; A61B 5/029; A61B 5/021; A61B 5/0205; A61B 5/02

(52) U.S. Cl. CPC A61B 5/7275; A61B 5/0205; A61B 5/02028; A61B 5/029; A61B 5/021; A61B 5/14542; A61B 5/742

(57) ABSTRACT

The invention provides a system to better detect the onset of congestive heart failure (CHF). The system consists of two primary components—the CHF Index Graph (CHFIG) and a body-worn sensor for measuring the necessary parameters—that correctly measure and analyze a set of parameters related to CHF. The CHFIG typically consists of 7 or 8 unique axes, each corresponding to a different CHF-related parameter. Each axis is normalized and scaled so that the plotted values trend outwards as a patient's trajectory towards CHF worsens. Thus, the area of a region constructed from the plotted values of the parameters increases as the values move further away from the center of the graph; this indicates the patient is trending towards CHF. Conversely, a healthy patient's parameters will be clustered relatively close to the center of the graph, creating a region with a much smaller area.

### CROSS-REFERENCE TO RELATED APPLICATIONS

This application claims benefit of U.S. Provisional Application No. 62/060,926, filed Oct. 7, 2014, which is hereby incorporated in its entirety including all tables, figures and claims.

### FIELD OF THE INVENTION

This invention relates to a graphical technique that plots a collection of physiological parameters to predict congestive heart failure (CHF).

### GENERAL BACKGROUND

The pathology that drives health failure, and particularly CHF, is complex. In general terms, CHF occurs when cardiac output (CO), the mathematical product of stroke volume (SV) and heart rate (HR), is insufficient in meeting the oxygen demands of the body. SV relates to volumetric pressure within the heart's right and left ventricles that stretch the underlying muscle cells comprising this tissue to their greatest physiological dimensions before contraction. This is called ‘preload’. Afterload is the tension or stress developed in the wall of the left ventricle during ejection. It relates to pressure in the aortic and pulmonary arteries, and more specifically the pressure that the ventricle must overcome to eject blood. Increased pressure in these arteries increases the afterload on the heart's ventricles. The ejection fraction (EF) represents the volumetric fraction of blood pumped out of the left ventricle with each heartbeat.

Causes of CHF are well known, and typically include coronary heart disease, diabetes, hypertension, obesity, smoking, and valvular heart disease. EF can be diminished (<50%), as in systolic heart failure, or normal (>65%), as in diastolic heart failure. The common characteristic of both forms is elevation of the pressure within the left atrium at the end of its contraction cycle, or left ventricular end-diastolic pressure (LVEDP). Chronic elevation of LVEDP causes transudation of fluid from the pulmonary veins into the lungs, resulting in shortness of breath (dyspnea), rapid breathing (tachypnea), and fatigue with exertion due to the mismatch of oxygen delivery and oxygen demand throughout the body. Early compensatory mechanisms include increased respiration rate (RR) and HR. As CO is compromised, the kidneys, due to their lack of perfusion, respond by retaining sodium and water, leading to an increase in intravascular volume. An early finding is an increase in fluids, which serves to estimate the LVEDP. As the LVEDP rises, pulmonary venous congestion worsens. Body weight increases incrementally and fluid may shift into the lower extremities. Medications for heart failure aim to interrupt the kidney's hormonal responses to diminished perfusion, and also work to help excrete excess sodium and water from the body. Nonetheless, this is an extremely delicate balance. Decompensated CHF can be driven by an increase in blood pressure (relating to afterload), or fluid retention (relating to preload), and/or a significant change in HR due to a tachyarrhythmia. A characteristic of this condition is a lack of response to oral medications. Thus admission to a hospital is often necessary for intravenous diuretic therapy.

CHF can be understood as a combination of deficiencies in eight physiological parameters, each independent of one another. When processed collectively, trends in these parameters may indicate the onset of CHF and whether or not a patient requires hospitalization. The most significant among these parameters are SV, systolic blood pressure (SYS), and thoracic fluid content (TFC). Additionally, blood oxygen (SpO2), HR, RR, CO and body weight also play a role in indicating the onset of CHF. Thus, in theory, both clinicians and patients should be able to analyze these parameters—SV, CO, SYS, TFC, SpO2, HR, RR, and body weight—to detect the onset of CHF.

Arguably the most important of these parameters, SV, relates to the volumetric pressures within the heart's left and right ventricles, and is indicative of the volume of blood that is pumped from the left ventricle with each heartbeat. The normal range for SV in healthy patients is somewhere between 55 ml and 100 ml. However patients with CHF typically have reduced contractility, which in turn lowers their SV. For example, a patient in need of hospitalization due to CHF typically has a SV of around 30-40 mL.

Similar to SV, SYS provides a significant indication of the onset of CHF. The chief determinants of SYS are the volume of blood being pumped into a vessel, along with the vessel's contractility or elasticity. CHF patients, as described above, typically feature a decreased SV, and thus a commensurate decrease in SYS. A recent study by Packer et al., the contents of which are incorporated herein by reference, indicated that decreasing SYS correlated well with the onset of CHF episodes and ultimately repeat hospital visits (Packer et al., “Utility of Impedance Cardiography for the Identification of Short-Term Risk of Clinical Decompensation in Stable Patients With Chronic Heart Failure Packer”, J Am Coll Cardiol. 6; 47(11), 2245-52 (2006)).

TFC is also an important parameter in identifying early stages of CHF. In CHF patients, the reduction in SV and CO diminishes perfusion to the kidneys. These organs then respond with a decrease in their filtering capacity, thus causing the patient to retain sodium and water, which leads to an increase in intravascular volume. This in turn, promotes congestion, which is manifested to some extent by a build-up of fluids in the patient's thoracic cavity (i.e. increased TFC). Such an increase in TFC, if significant enough, will ultimately lead to an increase in the patient's overall weight, which is why weight itself is often used as an indicator of CHF, albeit a relatively late one.

CHF onset can also be indicated by SpO2, which measures the amount of oxygen in the patient's blood and, ultimately, the perfusion of the patient's organs. A decrease in the level of blood pumped throughout the body means less oxygen reaches the vital organs. Therefore, SpO2 level tends to drop during the onset of CHF. This leads to compensatory increases in both HR and RR as the patient's cardio-pulmonary system attempts to increase oxygen flow to the vital organs.

Importantly, most of the above-specified parameters, taken on their own, have little degree of specificity for detecting CHF. For example, on any given day, a perfectly healthy person can display a similar increase in HR as a person undergoing CHF. However, collective analysis of a set physiological parameters, such as those described above, may predict the onset of CHF.

### SUMMARY OF THE INVENTION

Based on the above, the invention described herein involves analysis of a set of physiological parameters to detect CHF. The parameters, for example, can be SV, CO, SYS, TFC, HR, RR, SpO2, and body weight, each of which is known to trend either upward or downward with the onset of CHF. A clinician may interpret such trends to diagnose CHF. However, it can be difficult to simultaneously and quickly analyze multiple trends, especially when they correspond to different properties that may extend in opposing directions.

To cure this deficiency, the invention provides a system featuring a graphical technique to better detect the onset of CHF. The system consists of two primary components: the CHF Index Graph (CHFIG), and a body-worn sensor or multiple sensors for measuring the graph's necessary parameters. The CHFIG typically consists of 7 or 8 unique axes, each corresponding to a different CHF-related parameter. Each axis of the CHFIG is normalized and scaled so that the plotted values trend outwards (i.e. away from the graph's origin) as a patient's trajectory towards CHF worsens. A region can be constructed by connecting adjacent plotted values, wherein the area of the region increases as the plotted values indicate that the patient's CHF is worsening. Conversely, a healthy patient's parameters will be clustered relatively close to the graph's origin, thus resulting in a region with a relatively small area. To further simplify the identification of CHF, the region's color changes at different pre-determined thresholds, indicating the status of the patient in regards to CHF. The pre-determined thresholds can be determined with a clinical study conducted on a group of subjects, wherein the physiological parameters that indicate CHF's onset are determined from the study. As an example, the region is green when the patient is healthy, yellow with the onset of CHF, and red when the patient is entering a severe state of CHF, and ultimately risks hospitalization. In this way, both the clinician and the patient are able to examine the CHFIG and quickly and accurately diagnose the onset of CHF without having to independently analyze trends in 7 different plots.

In a preferred embodiment of the invention, a single body-worn sensor measures all physiological parameters used as input to the CHFIG, and the actual CHFIG is generated by a computer-controlled system. For example, the sensor can be worn like a conventional necklace, attaching to the patient's chest with two custom-fit electrodes. Using a collection of measurement technologies, the sensor can quickly measure SV, CO, SYS, TFC, HR, RR and SpO2 from the patient. Information describing these parameters is then sent wirelessly through Bluetooth® to a gateway system, e.g. a tablet computer, smartphone, or plug-in component such as the Qualcomm 2net system. The gateway system can process and display the information, along with the CHFIG. Additionally, the gateway system can forward the information to an accompanying web-based system for further review and additional display of the CHFIG. A remote clinician, for example, could review the CHFIG on the web-based system to determine if a patient is entering into CHF.

Patients of different demographics, including age, body weight, etc., will typically feature different values of physiological parameters when they are in a stable state, i.e. a state not representative of CHF. Such a stable state represents the patient's ‘baseline’, which is preferably collected before the CHFIG is generated. For example, while one patient may have a baseline SYS of 115 mmHg, another might show a value of 135 mmHg. Thus, in embodiments, the axes of the CHFIG are normalized with respect to the patient's stable baseline. Determining the baseline requires measurements for the above-mentioned physiological parameters when the patient is in a stable state. Once measured, this information will be stored and used as a precedent for determining a relative difference in future measurements. For example, if the patient SYS is 115 mmHg, this value lies at 0 on the CHFIG. Then the graph will plot the relative difference between the patient's current measurements, and their baseline measurements. Each axis is typically normalized. A line then connects the points on adjacent axes to form a region; the collective area of all regions on the CHFIG indicates the onset of CHF. In embodiments, a graphical user interface can display multiple CHFIGs, each corresponding to a different time period (e.g. a given day), that a clinician can quickly review to detect the onset of CHF. In response, the clinician can then instruct the patient to make slight adjustments to their diet, exercise, routine or even medication in order to prevent both physically and economically taxing re-hospitalization visits.

In one aspect, the invention provides a method for characterizing heart failure in a patient. The method includes the following steps: 1) using one or more sensors to generate a set of physiological data parameters including fluid level (e.g. TFC), SV, blood pressure (e.g. SYS), CO, and SpO₂; 2) transmitting the data parameters from the group of one or more sensors to a display device; and 3) generating a graphical image on the display device that shows a plot with multiple axes, each corresponding to a single data parameter, and organized so that a value corresponding to the data parameter is further separated from the plot's origin as the patient's state of heart failure worsens. The plot also displays a region calculated from the values plotted on each axis that increases in area as the patient's state of heart failure worsens.

In embodiments, the method generates a plot having an axis correlating to a single parameter that can be one of SV, CO, blood pressure, and SpO2. Here, the parameter's lower values are further removed from the origin compared to the higher values. In another embodiment, a first set of data parameters is collected when the patient is at a healthy, stable level (e.g. a baseline level). In further embodiments, a second set of data is collected after the first set. In another embodiment, the area of the figure created by the plotted data parameters increases as the patients state of CHF rate worsens. In still other embodiments, the plotted area changes color (e.g. from green to yellow to red) according to pre-determined thresholds that indicate the patient's state of CHF.

In another aspect, the invention provides a method for characterizing heart failure including the following steps: 1) using one or more sensors to generate a first set of data parameters including SV, CO, blood pressure, SpO2 and fluid level; 2) transmitting the first set of data parameters to a display device; 3) generating a second set of data parameters for SV, CO, blood pressure, SpO2, and fluid level, after the first set; 4) calculating a third set of data parameters from the first two sets, that indicates change in a data parameter, calculated by finding the difference between the first and second sets data parameters; and 4) generating a graphical image on the display device that shows a plot with multiple axes, each corresponding to a single change in data parameter generated by a sensor, and organized such that the value corresponding to change in a data parameter is further separated from the graph's origin as the patient's state of heart failure worsens. The region calculated from the values plotted on each axis increases in area as the patient's state of heart failure worsens.

In embodiments, the method generates a plot having an axis correlated to the change in a single data parameter that can be one of SV, CO, blood pressure, SpO2, or fluid level, in which the lower values of the change for each data parameter are further separated from the graph's origin. In another embodiment, the region calculated from the values plotted on each axis increases as the patient's state of heart failure worsens. In further embodiments, the region is shaded with a color that changes color according to pre-determined thresholds designed to indicate the patient's state of heart failure. In different embodiments, the display device may be a mobile device, or computer.

In another aspect, the invention provides a method for characterizing heart failure which includes the following steps: 1) using one or more sensors to generate a first set of data parameters including SV, CO, blood pressure, SpO2 and fluid level; 2) transmitting the first set of data parameters to a display device; 3) generating a second set of data parameters for SV, CO, blood pressure, SpO2, and fluid level, after the first set; 4) transmitting the second set of data parameters to a display device; 5) generating a third set of data parameters indicating a change in a particular parameter by subtracting the first set from the second set; 6) determining a maximum value for each of the data parameters in the third set; 7) normalizing the third set of data parameters for each of the data parameters such that they can fit on graphical axes having comparable dimensions; 8) generating a graphical image on the display device that shows a plot with multiple axes, each corresponding to a single normalized change in data parameter generated by a sensor, and is organized such that the value corresponding to normalized change in a data parameter is further separated from the graphs origin as the patient's state of heart failure worsens; the region calculated from the values plotted on each axis also increases in area as the patients state of heart failure worsens; 9) calculating the area of the region created from the values plotted on each axis using a mathematical algorithm; and 10) generating another graphical image on the display device that shows a two axis plot with a line created from the areas calculated that is organized such that the slope increases as the patient's state of heart failure worsens.

In one embodiment, the horizontal axis of the plot corresponds to the date that the measurement was taken. In another embodiment, the vertical axis of the plot will correspond to the calculated CHF index number.

In another embodiment, the invention provides a method for calculating the area of the region created from the data parameter values plotted on each axis, including the following steps: 1) decomposing the figure into a series of triangles wherein two adjacent axes create two sides (e.g. side A and side B) of the triangle, and the line connecting the two values plotted on the axes creates another side (e.g. side C) of the triangle; 2) determining the angle at the origin (e.g. angle a); 3) using the function: ½ AB×sin(a) to determine the area of each of the triangles; and 4) calculating the sum of the areas of each of the individual triangles to determine the area of the entire figure. This value represents an index that can estimate the onset of CHF. Typically, an increase in value indicates an increase in the severity of the CHF.

Still other embodiments are within the scope of the drawings and detailed description, below.

### BRIEF DESCRIPTION OF THE DRAWINGS

Embodiments of the invention will now be described in greater detail in conjunction with the figures, in which:

FIG. 1 is a schematic drawing showing a system of a preferred embodiment of the invention used to generate a collection of CHFIGs;

FIG. 2 is a 7-axis CHFIG for a single measurement indicating that the patient's CHF level is severe;

FIG. 3 is a 7-axis CHFIG for a single measurement indicating that the patient's CHF level is moderate;

FIG. 4 is a 7-axis CHFIG for a single measurement indicating that the patient's CHF level is stable;

FIG. 5 is an illustration of a single region of the CHFIG, similar to those shown in FIGS. 2-4, complete with a formula used to calculate the region's area;

FIG. 6 is a scatterplot showing the area of a CHFIG, similar to that shown in FIGS. 2-4, plotted as a function of time;

FIG. 7 is a front view of a preferred sensor, worn around a patient's neck, for measuring physiological parameters representing input for a CHFIG; and

FIG. 8 is a schematic drawing showing a system of an alternate embodiment of the invention used to generate a collection of CHFIGS.

### DETAILED DESCRIPTION

Generating a CHFIG

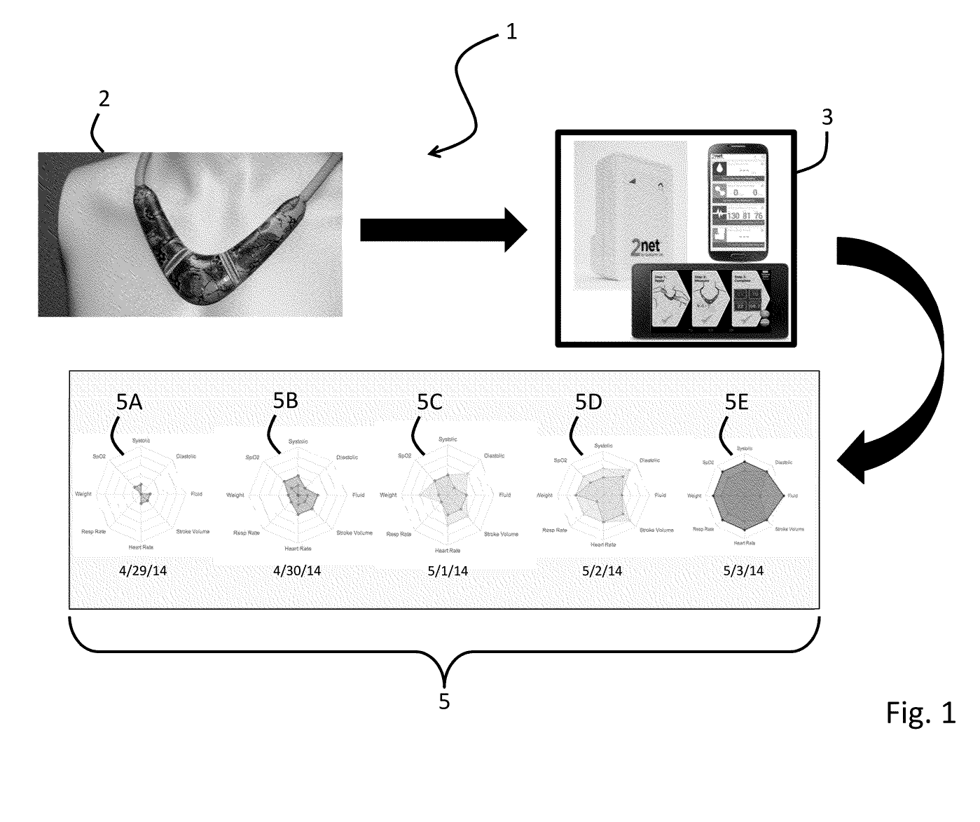

FIG. 1 shows a system 1 used to generate a collection 5 of CHFIGs 5A-E according to the invention. The collection 5 shows individual CHFIGs measured over a 5-day period. The system 1 features a body-worn sensor 2 that measures physiological parameters, e.g. SV, CO, SYS, TFC, SpO2, HR, RR, that represent input for the CHFIGs 5. An example of the body-worn sensor 2 is described in detail in the following co-pending patent application, the contents of which are incorporated herein by reference: NECK-WORN PHYSIOLOGICAL MONITOR, U.S. Ser. No. 62/049,279, filed Sep. 11, 2014. Such a sensor is also summarized briefly, below. After the sensor 2 measures the physiological parameters, it wirelessly transmits them to a gateway system 3, which is typically a tablet computer, smartphone, or Qualcomm 2net system. Some versions of the gateway system 3, such as the tablet computer or smartphone, display the physiological parameters and corresponding CHFIG 5. In embodiments, the gateway system 3 forwards the physiological parameters to a web-based system, which then displays them accordingly.

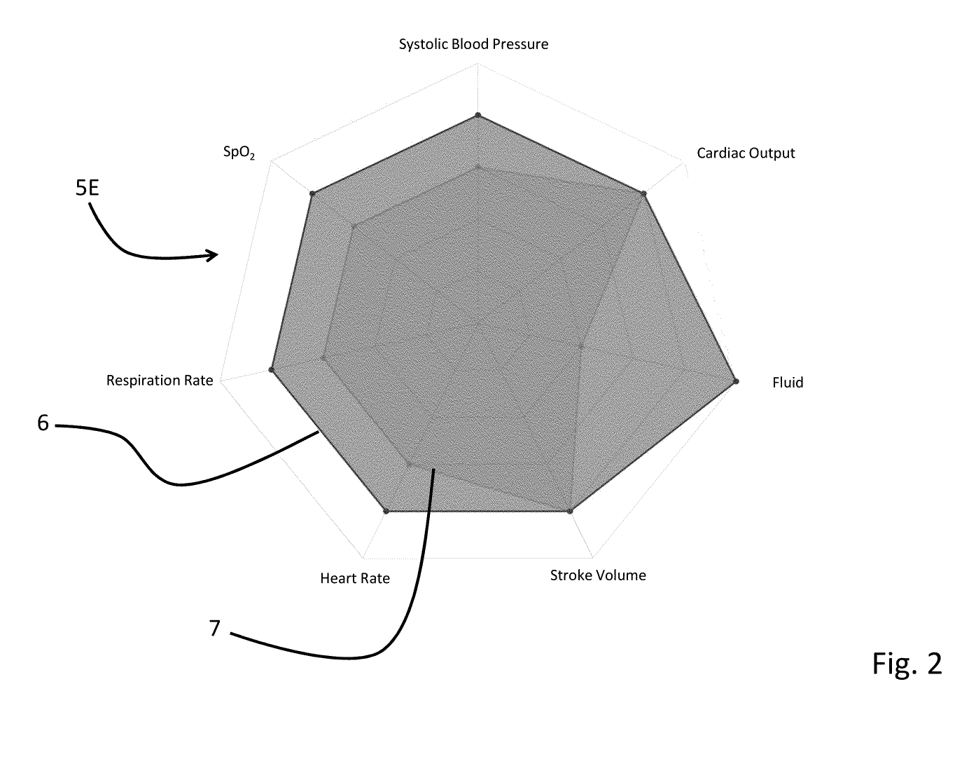

FIG. 2 shows an example CHFIG 5E taken from the collection 5 of CHFIGs shown in FIG. 1. The CHFIG 5E includes 7 unique axes, each corresponding to a unique physiological parameter measured by the sensor 2 of FIG. 1. The CHFIG 5E is constructed such that each axis is ‘normalized’ according to a baseline measurement made while the corresponding patient is in a stable condition, as described above. In one embodiment, ‘normalized’ means that the axis is constructed so that its value at the CHFIG's origin corresponds to a value of ‘0’, and its value at the axis's maximum corresponds to an arbitrary value of ‘100’. Other mathematical techniques for normalizing the CHFIG's axis can also be used. Importantly, the CHFIG is constructed so that when the normalized value of the physiological parameter is plotted on its corresponding axis, its separation from the axis's origin increases as the value of the physiological parameter indicates that the patient's CHF condition is worsening. For example, it is well known that a decrease in SV indicates the onset of CHF. Thus, for the CHFIG 5E, the axis labeled ‘stroke volume’ has larger values closer to the origin, and lesser values further away from the origin. Conversely, it is well know that fluid values (e.g. TFC) typically increase with the onset of CHF. Thus, the axis labeled ‘fluid’ is oriented in the opposite manner as the axis labeled ‘stroke volume’, i.e. it features lesser values closer to the origin, and greater values further away from the origin.

Each CHFIG 5E features two separate regions 6, 7. The first region 6 is drawn from physiological values measured from the current day, and is colored either red, green, or yellow depending on the severity of the patient's CHF. The second region 7 is drawn from physiological values measured from the day before the current day, and is shaded gray. Simultaneous display of both regions 6, 7 allows the viewer to quickly assess the progression of the patient's degree of CHF: if the region 7 corresponding to the current day has a larger area than the region 6 corresponding to the earlier day, then the patient's CHF is worsening; conversely, the opposite scenario indicates that the patient's CHF is improving.

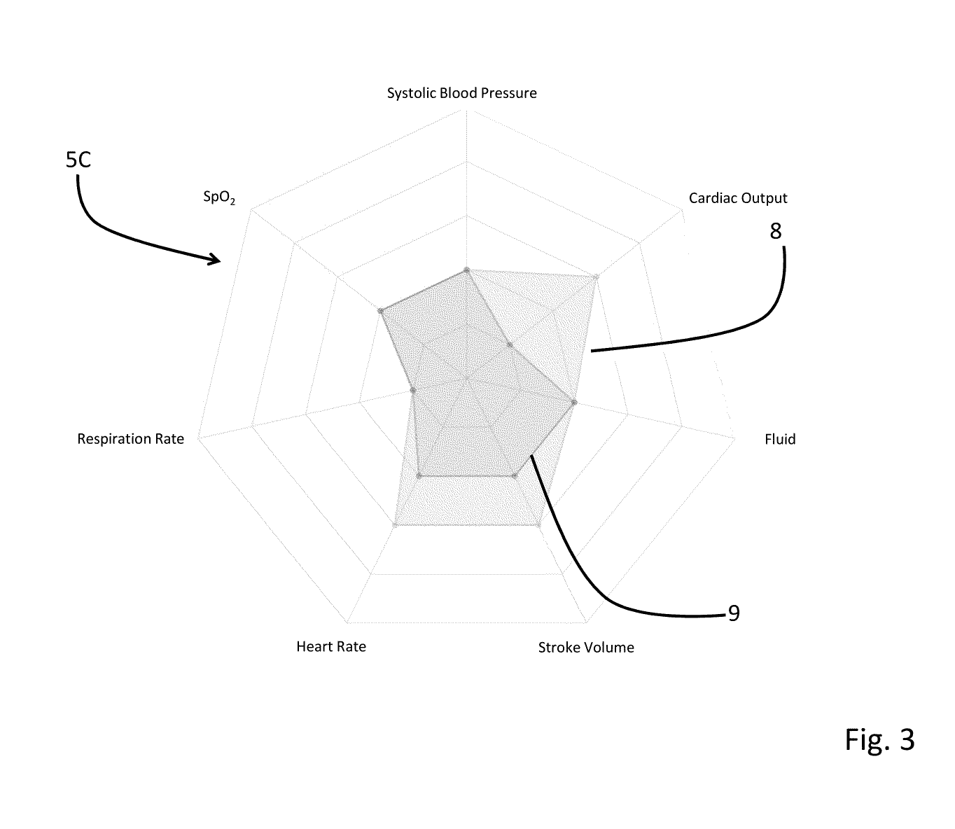

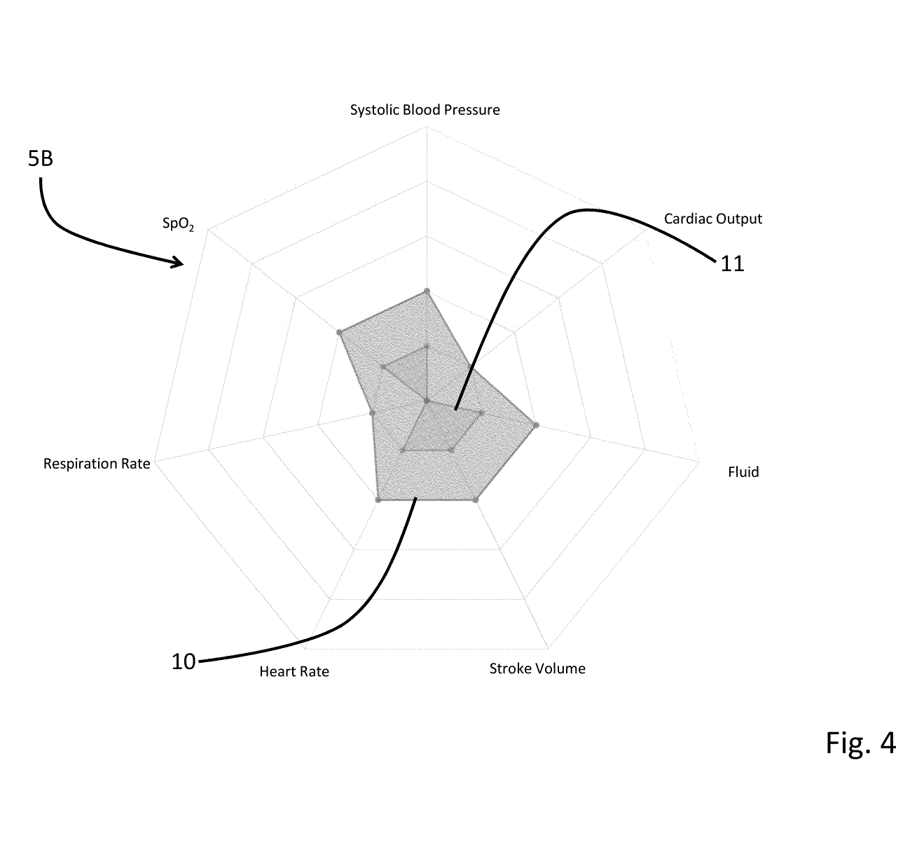

FIG. 3 shows a similar CHFIG 5C featuring two separate regions 8, 9 similar to those described above with reference to FIG. 2. Here, the CHFIG 5C is colored yellow, indicating the patient's CHF is in a moderate state. And FIG. 4 shows another similar CHFIG 5B featuring similar regions 10, 11. The CHFIG 5B is colored green, indicating the patient's CHF is in a stable state.

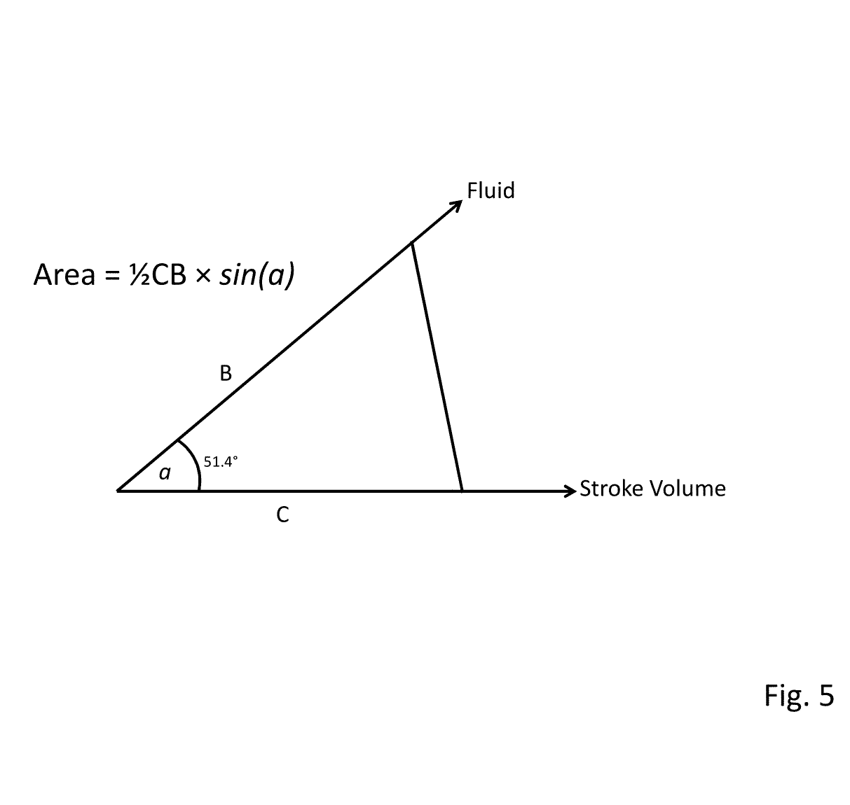

FIG. 5 shows how the area of a graphical region, such as regions 8 and 9 described above, are calculated for a particular CHFIG. More specifically, the graph can be broken down into a series of triangles, two sides of which is formed by adjacent axes, and one formed by the line connected the two neighboring points on each axis. The center angle is known, and is calculated simply by dividing 360° by the number of axes. Once this is determined, the area of the triangle is calculated with the following equations:

For 7 Axes:Area=½CB×sin(51.4°)

For 8 Axes:Area=½CB×sin(45°)

This calculation is performed for all of the adjacent triangles within the region. Once the area for each of the adjacent triangles has been calculated, the sum of the calculated areas indicates the CHF Index Number (CHFIN), representing the area of the entire region.

In one embodiment, the relative contribution of each parameter to the CHFIG is the same, i.e. they are weighted equally. Alternatively, the contributions of certain parameters that are known to be superior indicators of CHF are weighted in an increased manner. For example, as described above, parameters such as SV, SYS and TFC are stronger indicators of the onset of CHF. Thus in some embodiments, the areas corresponding to these values may be given an increased weight when calculating the CHFIN.

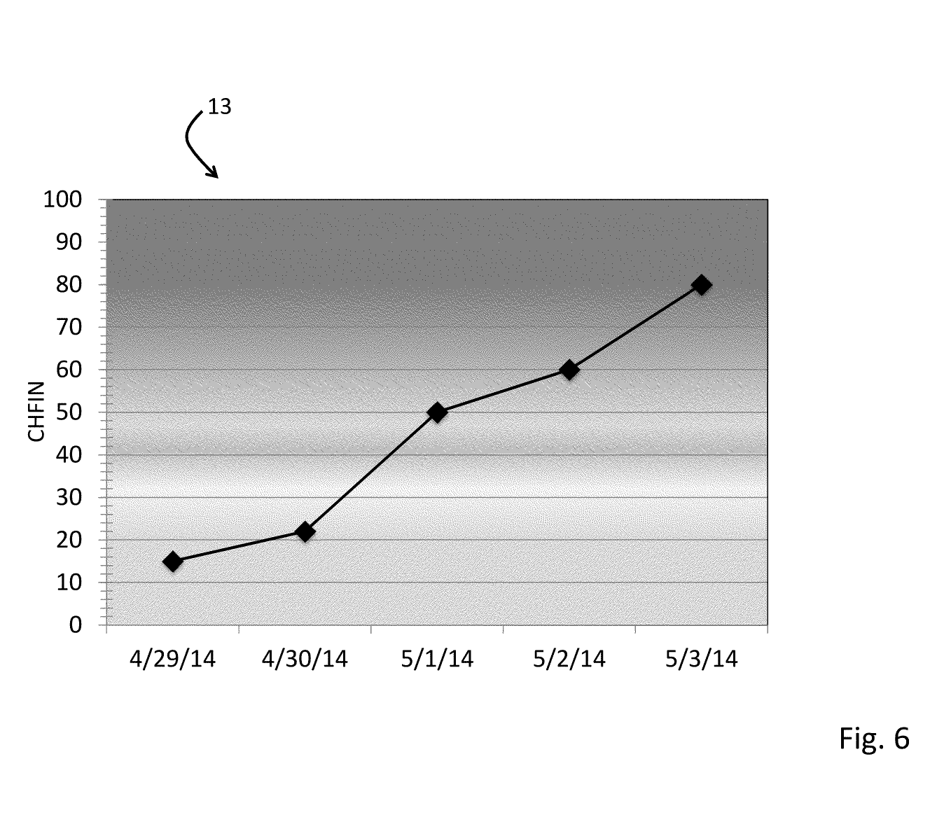

Once the CHFIN is calculated, it can be plotted in a conventional scatterplot format, such as that shown in FIG. 6. Here, the scatterplot 13 shows time-dependent values of the CHFIN. The scatterplot can be colored in a manner that is commensurate with the CHFIGs, i.e. green regions indicating that the patient is in a relatively healthy state, yellow indicating their CHF is worsening, and red indicating that the CHF is severe.

Converting Physiological Measurements to Graphical Data

As described above, a baseline measurement is typically required to generate a CHFIG. Here, to generate the baseline, the system records measurements for each of the 7 or 8 parameters when the patient is in a relatively stable state (i.e. a state indicated in the CHFIG by the a green color). After these measurements have been recorded and stored, all future measurements made by the system depict the relative difference between their current measurement and their stable measurement.

Measurements plotted on each axis are normalized, and plotted so that their values extend from the axis's origin as they indicate a worsening of CHF. For SV, for example, values will decrease as a patient begins trending towards CHF. Typically, a healthy patient has a SV of 70 mL or greater, while a patient entering CHF will have a SV about 30 mL. SYS is similar to SV in that it also decreases as a patient is nearing CHF. A typical SYS blood pressure is between 140 mmHg and 100 mmHg, while a patient who is entering CHF can drop to 80 mmHg or lower. Fluid levels, as indicated by TFC, will rise as a patient undergoes a CHF episode. The standard units for measuring fluid levels are Ohms, a unit that is inversely related to the actual fluid levels within the patient. Thus the lower the number in Ohms, the higher the fluid content within the patient. A normal healthy, hydrated person will have a TFC of about 19 Ohms. A patient entering CHF who has begun retaining an excess amount of fluid can have a TFC of approximately 11 Ohms or lower.

SpO2 is also an important consideration in identifying the onset of CHF. SpO2 is measured on a scale of percent blood oxygenation, with the maximum being 100%. A normal healthy level is roughly 97%-98%, while someone with CHF can dip below 85%. CO can also indicate the onset of CHF. A healthy patient will have a CO of about 3.5-6 L, while someone approaching CHF might only have a CO of 2.5 L.

HR and RR both will go up as the patient is entering into CHF. These two parameters, however, are less indicative of CHF than the others because there are so many different reasons a patient's HR and RR can rise (i.e. rapid movement, stress, etc.) Nonetheless, consistently high values of HR and RR may indicate the onset of CHF, especially when these values are collectively analyzed with the parameters described above. A normal resting HR is about 50-60 beats/minute, while a normal RR is about 7-12 breaths/minute. A person entering CHF however, can have a HR greater than 150 beats/min, while their RR can reach over 20 breaths/minute.

Other parameters, such as weight, can be plotted in the CHFIG as a separate axis.

Sensor

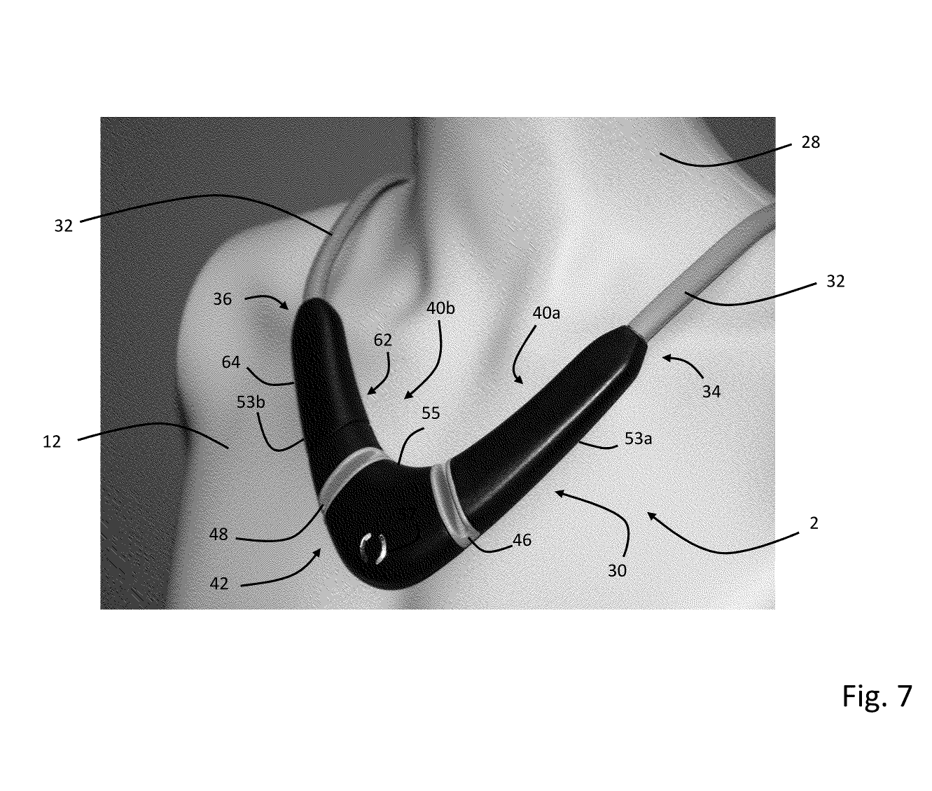

FIG. 7 shows an embodiment of a body-worn sensor 2, according to the invention, that can used to measure physiological parameters for the CHFIG. A patient 12 wears the sensor 2 around their neck 28 so that it rests against the sternum, similar to a necklace or other neck-adorning jewelry. The sensor 2 features a sensing portion 30 and a securement member 32 (or securement members in an alternate embodiment, not illustrated). As illustrated, the securement member 32 extends from a first end 34 of the sensing portion 30 and attaches to a second end 36 of the sensing portion 30. The securement member 32 is long enough to pass behind the patient's neck 28 and to hold the sensing portion 30 in proper position for sensing electrodes attached to its rear, patient-facing surface to be attached to the proper locations on the patient's chest. This ensures that the sensing portion 30 is placed in approximately the same position for each measurement made on a particular patient, and that it is held in proper position to acquire the relevant bioelectric signals, as explained more fully below. Additionally, the securement member 32 houses a battery in battery compartment (not shown), which is positioned generally in the middle of the securement member 32 (lengthwise speaking) such that it is positioned inconspicuously behind the patient's neck 28 when the sensor 2 is worn.

In other, non-illustrated embodiments, the securement member could be split in the middle, with flexible yet shape-retaining “branches” extending from the first and second ends 34, 36 of the sensing portion 30 so as to pass behind the patient's neck 28, but not connect, much like a physician's stethoscope. In that case, the battery compartment could be located in one of the branches or, alternatively, in the sensing portion 30 of the sensor 2. In still further non-illustrated embodiments, a securement member might not be included, in which case attachment of the electrodes to the patient's body would, by itself, be used to hold the sensor in position. Ultimately, however, where a securement member is provided to facilitate positioning of the sensing portion 30 on the patients' body, what is important is simply that the securement member should be configured to pass at least substantially around the patient's neck 28 (which includes a configuration in which lateral halves of the securement member pass posteriorly over the trapezius muscles without curving medially toward the spine). In other words, the securement member 32 passes sufficiently over the trapezius region and/or behind the neck to support the sensing portion 30 and prevent it from falling before the sensing portion 30 is secured to the patient's body via the electrodes, as described more fully below.

The sensing portion 30 is constructed in two or more sections or segments, e.g. a central segment 42 and two outboard segments 40a and 40b, to the rear of which electrode patches are attached as described below. The electrode patches perform thoracic bio-impedance (TBI) and electrocardiogram (ECG) measurements, as described in more detail below. The segments are connected to each other by means of flexible connector segments, which in turn are encased in flexible housing 46 and 48. The flexible connector segments are typically made from a polymeric material, e.g. Kapton® flexible printed circuits available from the DuPont Corporation. Such materials are essentially a flexible, polymeric film that encases one or more thin conducting members, which are typically made from copper. Each of the segments 40a, 40b, and 42 includes, respectively, a rigid circuit board populated with discrete electrical circuit components, described in more detail below. The rigid circuit boards connect to one another via the flexible connector segments, which each include about 20 conductive members.

Suitably, the connector segments 46 and 48, which may be formed as rubber boots designed to snap into respectively opposing ends of the protective housing segments, are typically made from soft, flexible material such as silicone rubber. Generally speaking, such a configuration of the sensing portion 30 serves to hold the sensing electrodes at their proper positions before they are adhered to the patient's chest, while allowing the sensing portion 30 to conform to the different curvatures of the physiological region upon which it rests.

The sensor measures the following parameters from a patient: HR, PR, SpO2, RR, TTC, SV, CO, and a parameter sensitive to blood pressure (e.g. SYS) called pulse transit time (PTT). From SV, a first algorithm employing a linear model can estimate the patient's pulse pressure (PP). And from PP and PTT, a second algorithm, also employing a linear algorithm, can estimate SYS and DIA. Thus, the sensor, acting alone, can measure all five vital signs (HR/PR, SpO2, RR, TEMP, and SYS/DIA) along with hemodynamic parameters (SV, CO, TFC).

To measure these properties, disposable electrodes attach directly to the sensor to secure it in close proximity to the patient's body without bothersome cables. In particular, the electrodes are provided in patches, with each electrode patch containing two electrode regions to measure ECG and TBI waveforms. The above-referenced patent application entitled NECK-WORN PHYSIOLOGICAL MONITOR, which has previously been incorporated herein by reference, describes how these signals are measured. The patches easily connect to circuit boards contained within the sensor by means of magnets that are electrically connected to the circuit boards, to provide signal-conducting electrical couplings. Prior to use, the electrodes are simply held near the circuit boards, and magnetic attraction causes the electrode patches to snap into proper position, thereby ensuring proper positioning of the electrodes on the patient's body.

With light-emitting diodes operating in the red (e.g. 600 nm) and infrared (e.g. 900 nm) spectral regions on its chest-facing surface, the sensor measures SpO2 and pulse rate (PR) by pressing lightly against capillary beds in the patient's chest. Operating in a reflection-mode geometry, the sensor measures photoplethysmogram (PPG) waveforms with both red and infrared wavelengths. SpO2 is processed from alternating and static components of these waveforms. PR, in turn, can be calculated from neighboring pulses, typically from the PPG waveform generated with infrared light, as this typically has a relatively high signal-to-noise ratio.

All analog and digital electronics associated with these various measurements are directly integrated into the sensor. This means that a single, unobtrusive component—shaped like a piece of conventional jewelry instead of a bulky medical device—measures a robust set of parameters that can characterize a patient using both one-time and continuous measurements. Measurements can take place over just a few minutes or several hours, and can be made in medical facilities and at home. The sensor includes a simple LED in its base (i.e. sensing) portion, which is located near the center of the chest when worn by the patient. The sensor also includes a wireless transmitter (operating Bluetooth® and/or 802.11a/b/g/n) than sends data to a gateway system, as described above.

The sensor also includes a motion-detecting accelerometer, from which it can determine motion-related parameters such as posture, degree of motion, activity level, respiratory-induced heaving of the chest, and falls. The sensor can operate additional algorithms to process the motion-related parameters to measure vital signs and hemodynamic parameters when motion is minimized and below a pre-determined threshold, thereby reducing artifacts. Moreover, the sensor estimates motion-related parameters such as posture to improve the accuracy of calculations for vital signs and hemodynamic parameters.

The sensor measures all of the above-mentioned properties while featuring a comfortable, easy-to-wear form factor. It is lightweight (about 100 grams) and battery-powered. The sensor's form factor is designed for comfort and ease of use, with the ultimate goal of improving patient compliance so that the above-mentioned parameters can be measured in a continuous manner and on a day-to-day basis. The system is targeted for elderly, at-home patients, e.g. those suffering from chronic conditions such as HF, CHF, ESRD and related cardiac diseases, diseases of the kidneys, diabetes, and chronic obstructive pulmonary disease (COPD).

### OTHER EMBODIMENTS

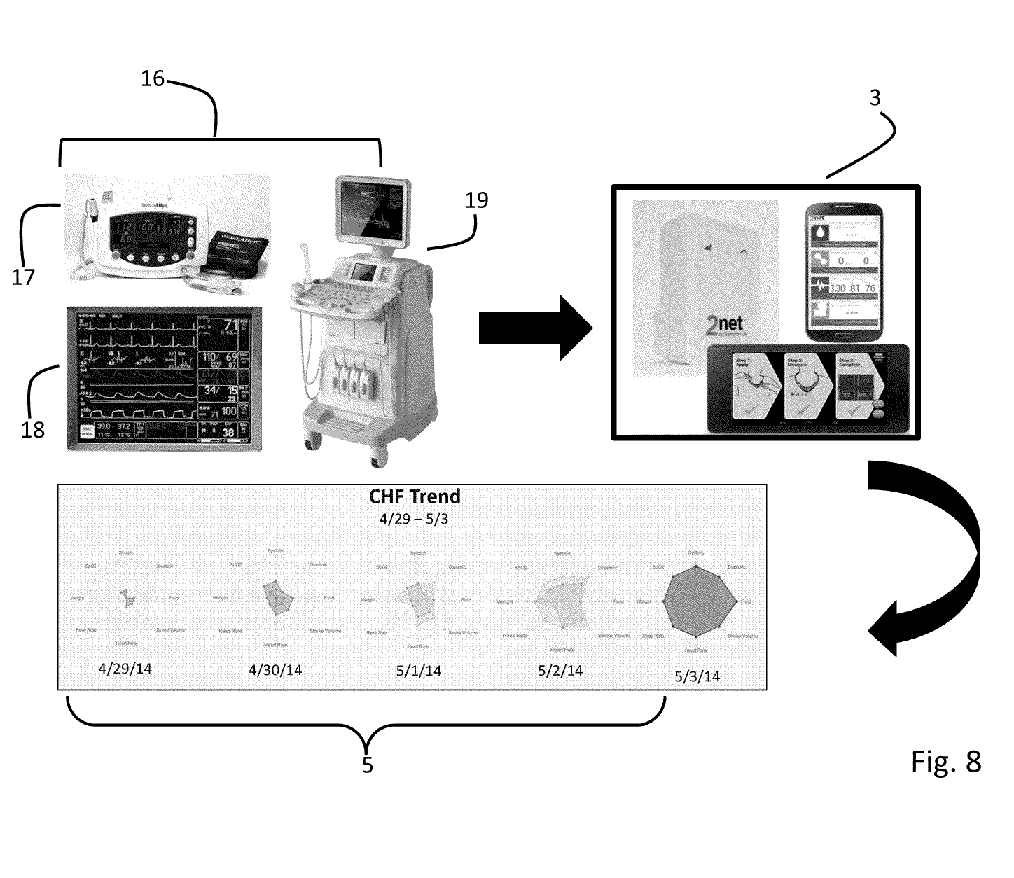

Other embodiments of the invention are possible. In particular, referring to FIG. 8, devices 16 other than the sensor shown in FIG. 7 can generate the physiological parameters required to construct a CHFIG. For example, a conventional blood pressure cuff 17, or alternatively a radial arterial catheter, can be used to generate blood pressure values such as SYS. A conventional pulse oximeter can be used to measure SpO2. For SV and CO, an impedance cardiography machine, Doppler ultrasound machine 19, or a pulmonary arterial catheter can be used. The impedance cardiography machine or alternatively a chest x-ray can be used to measure TFC. A conventional vital sign monitor 18 can also be used to measure HR and RR. And as mentioned above, a conventional Bluetooth-enabled scale can be used to measure weight if this parameter is included in the CHFIG.

Such devices may transmit values corresponding to the parameters through a wireless interface associated with the system. Alternatively, these values can be entered manually.

Still other embodiments are within the scope of the following claims.

What is claimed is:

1. A method for characterizing heart failure in a patient, comprising the following steps:
generating the following data parameters with a group of one or more sensors: fluid level, stroke volume, blood pressure, cardiac output, SpO2;
transmitting the data parameters from the group of one or more sensors to a display device;
generating a graphical image on the display device, the graphical image showing a plot comprising: i) multiple axes, with each axis corresponding a single data parameter generated by a sensor in the group of one or more sensors, and organized so that a value corresponding to the data parameter is further separated from the plot's origin as the patient's state of heart failure worsens; and ii) a region calculated from values plotted on each axis that increases in area as the patient's state of heart failure worsens.

2. The method of claim 1, wherein the plot comprises an axis corresponding to stroke volume with lower values of stroke volume being further separated from the graph's origin.

3. The method of claim 1, wherein the plot comprises an axis corresponding to blood pressure with the lower values of blood pressure being further separated from the graph's origin.

4. The method of claim 1, wherein the plot comprises an axis corresponding to fluid level with the higher values of fluid levels being further separated from the graph's origin.

5. The method of claim 1, wherein the plot comprises an axis corresponding to cardiac output with the lower values of cardiac output being further separated from the graph's origin.

6. The method of claim 1, wherein the plot comprises an axis corresponding to SpO2 with the lower values of SpO2 being further separated from the graph's origin.

7. The method of claim 1, further comprising the step of collecting and recording a first set of data parameters from the patient when the patient is in a healthy, stable level.

8. The method of claim 7, further comprising the step of collecting a second set of data parameters, the second set collected after the first set.

9. The method of claim 1, wherein the area of the region calculated from values increases as the patient's state of heart failure worsens.

10. The method of claim 9, wherein the region is shaded with a color that changes from green to yellow to red to indicate the patient's state of heart failure.

11. The method of claim 10, wherein the color changes when the area exceeds a pre-determined numerical threshold.

12. The method of claim 1, wherein the display device is a mobile device.

13. The method of claim 1, wherein the display device is a computer.

14. The method of claim 1, wherein the information is manually plotted and displayed on paper.

15. A method for characterizing heart failure in a patient, comprising the following steps:
generating the following first set of data parameters with a group of one or more sensors: fluid level, stroke volume, blood pressure, cardiac output, SpO2, the first set of data parameters measured from the patient when the patient is in a relatively healthy state;
transmitting the first set of data parameters from the group of one or more sensors to a display device;
generating the following second set of data parameters with a group of one or more sensors: fluid level, stroke volume, blood pressure, cardiac output, SpO2, the second set of data parameters measured from the patient after the first set of data parameters;
calculating a third set of data parameters from the first and second set of data parameters, the third set of data parameters indicating a change in a data parameter and calculated by collectively processing the first and second set of data parameters;
generating a graphical image on the display device, the graphical image showing a plot comprising: i) multiple axes, with each axis corresponding to a single change in a data parameter generated by a sensor in the group of one or more sensors, and organized so that a value corresponding to the change in the data parameter is further separated from the plot's origin as the patient's state of heart failure worsens; and ii) a region calculated from values plotted on each axis that increases in area as the patient's state of heart failure worsens.

16. The method of claim 15, wherein the plot comprises an axis corresponding to a change in stroke volume, with lower values of the change in stroke volume being further separated from the graph's origin.

17. The method of claim 15, wherein the plot comprises an axis corresponding to a change in blood pressure, with lower values of the change in blood pressure being further separated from the graph's origin.

18. The method of claim 15, wherein the plot comprises an axis corresponding to a change in cardiac output, with lower values of the change in cardiac output being further separated from the graph's origin.

19. The method of claim 15, wherein the plot comprises an axis corresponding to a change in fluid levels, with lower values of the change in fluid levels being further separated from the graph's origin.

20. The method of claim 15, wherein the plot comprises an axis corresponding to a change in SpO2, with lower values of the change in SpO2 being further separated from the graph's origin.

21. The method of claim 15, wherein the area of the region calculated from values of data parameter set three increases as the patient's state of heart failure worsens.

22. The method of claim 21, wherein the region is shaded with a color that changes from green to yellow to red to indicate the patient's state of heart failure.

23. The method of claim 15, wherein the display device is a mobile device.

24. The method of claim 15, wherein the display device is a computer.

25. The method of claim 15, wherein the information is manually plotted and displayed on paper.
